(19) United States
(12) Patent Application Publication
(10) Pub. No.: US 2016/0304577 A1
(43) Pub. Date: Oct. 20, 2016

(54) GLYCOSYLATED GLYCOPHORIN PEPTIDES

(71) Applicant: GLYCOTOPE GMBH, Berlin (DE)

(72) Inventors: Steffen GOLETZ, Berlin (DE); Christoph GOLETZ, Berlin (DE)

(21) Appl. No.: 15/102,186

(22) PCT Filed: Dec. 11, 2014
(86) PCT No.: PCT/EP2014/077314

§ 371 Date: Jun. 6, 2016

(30) Foreign Application Priority Data
Dec. 11, 2013 (EP) 13196653.3

(51) Int. Cl. C07K 14/705; A61K 47/48

(52) U.S. Cl. CPC C07K 14/705; A61K 47/48276

(57) ABSTRACT

The present invention pertains to glycosylated peptides of the glycophorin protein and their use in medicine. In particular, the peptides carry a carbohydrate structure of interest and are capable of binding to and being presented by MHC proteins. Using the glycosylated glycophorin peptides, a specific immune response against the carbohydrate structure of interest can be induced.

### FIELD OF THE INVENTION

The present invention pertains to glycosylated peptides of the glycophorin protein and their use in medicine. In particular, the peptides carry a carbohydrate structure of interest and are capable of binding to and being presented by major histocompatibility complex (MHC) proteins. Using the glycosylated glycophorin peptides, a specific immune response against the carbohydrate structure of interest can be induced.

### BACKGROUND OF THE INVENTION

Aberrant glycosylation is a typical hallmark of cancer cells. Carbohydrate tumor antigens on glycoproteins and glycolipids are therefore targets for active and passive immunotherapy. These highly abundant antigens are de novo expressed or upregulated due to changes in the complex glycosylation apparatus of tumor cells. Various lipid or protein bound carbohydrate tumor antigens are described, e.g. GM2, GD2, GD3, fucosylated GM1, Globo H, Le^{y} and the mucin core structures Tn, Sialyl-Tn and the Thomson Friedenreich antigen.

For example, the Thomsen-Friedenreich antigen alpha (TFα) is a known carbohydrate structure described as a tumor antigen in a series of reports. TFα is the disaccharide Gal-β1,3-GalNAc which is O-glycosidically linked in an alpha-anomeric configuration to the hydroxy amino acids serine or threonine of proteins in carcinoma cells. The core-1 carbohydrate structure motif corresponds to TFα and is present in many O-glycans of naturally occurring glycoproteins. However, in healthy and benign-diseased tissue core-1 forms the central core of the carbohydrate structure and carries further saccharide units which mask the core-1 structure. In a majority of carcinomas and in some non-epithelial malignancies, however, the core-1 structure is uncovered, forming the TFα antigen. Therefore, TFα is a specific pan-carcinoma antigen.

TFα is an important tumor antigen. TFα is expressed on over 60% of primary colon carcinomas and over 90% of liver metastases from colon cancer as well as on the majority of the carcinomas of other major indications including breast, lung, ovarian, prostate, and other gastrointestinal cancers such as gastric, and pancreatic carcinomas. TFα is an independent prognostic marker for patients with colon carcinomas, the mortality rate increases and the medium survival decreases in accordance with the increasing intensity of TFα expression. The development of liver metastases correlates with the expression of TFα. Patients with TFα positive primary carcinomas develop liver metastases in nearly 60% of the cases, while the risk for liver metastasis with TFα-negative tumors is significantly lower (less than 20%). Besides mediating metastasis into the liver, TFα may also play a role in the metastasis via the endothelium.

The exceptionally high pan-carcinomic specificity, prognostic relevance and direct involvement in liver metastasis nominate TFα as a prime target for cancer immunotherapy. There were attempts to provide a therapy approach based on Thomsen-Friedenreich. E.g. Shigoeka et al. (1999) describe the inhibition of liver metastasis from neuramidase treated Colon 26 cells by an anti-Thomsen-Friedenreich specific monoclonal antibody in a mouse model. However, due to the difficulties in generating highly specific anti-TF antibodies and because of their nature as IgM isotypes with comparably lower intrinsic affinities of single binding domains, TF-specific antibodies were not further developed so far. Further, some anti-TF-antigen antibodies are not clinically useful because they cause undesirable proliferation of tumor cells. Also WO 2006/012626 describes the therapeutic use of anti-TF antigen antibodies. Binding of TF-specific antibodies has been shown to inhibit the proliferation of tumor cells (Jeschke et. al. (2006)).

Furthermore, there were also attempts to develop vaccines based on Thomsen-Friedenreich. Most of them focused on the induction of antibody responses. E.g. Livingston and Lloyd (2000) used non-natural TF-conjugates, wherein synthetic TF was randomly coupled to KLH. These conjugates raised a humoral immune response against synthetic TF but not against TF on natural ligands (Adluri et al. (1995)). They were thus not TF specific as they would not recognize TF on a tumor structure.

Springer and Desai used vaccination with a T/Tn vaccine composed of types O and MN red blood cell derived glycoproteins which resulted in improved breast cancer patient survival, although only small amounts of IgM were made. However, IgM represents a less mature immune response and many previous studies relating to antibodies to TF-Ag involve IgM antibodies, therefore more pronounced highly TF specific immune responses would be needed and preferably an IgG response.

Therefore, it is the object of the present invention to provide alternative TFα vaccines which are capable of inducing a specific immune response against TFα.

### SUMMARY OF THE INVENTION

The present inventors found that glycosylated peptides of glycophorin carrying a carbohydrate structure of interest are presented by antigen presenting cells. These antigen presenting cells loaded with the glycosylated glycophorin peptide can then activate T cells against the carbohydrate structure of interest. Thereby, it was demonstrated that these glycosylated glycophorin peptides can induce a specific cellular immune response against the carbohydrate structure of interest. In particular, glycophorin peptides glycosylated with a tumor-associated carbohydrate antigen, such as TFα or Tn, are useful for vaccination against or treatment of cancer presenting said tumor-associated carbohydrate antigen. Since the glycophorin from which the peptides are derived is of human origin and naturally comprises carbohydrate structures, the peptide part of the glycosylated peptide has a very low risk of being immunogenic in humans. Therefore, any immune response against the glycosylated glycophorin peptide should exclusively be directed against the carbohydrate structure of interest. Thereby, undesired side effects, in particular undesired immune responses against the peptide backbone are highly improbable.

Hence, in a first aspect, the present invention provides a glycosylated glycophorin peptide comprising
(i) at least one amino acid independently selected from serine and threonine which carries a carbohydrate structure; and
(ii) at least 5 consecutive amino acids which are identical to or have at least 75% homology to an amino acid segment of the same length within the amino acid sequence of SEQ ID NO:1.

The glycosylated glycophorin peptide in particular is capable of binding to MHC class II proteins and the carbohydrate structure in particular has the formula Galβ1,3-GalNAcα1-.

In a second aspect, the present invention provides a conjugate comprising the glycosylated glycophorin peptide according to the first aspect of the invention covalently coupled to another agent.

In a third aspect, the present invention provides a method for producing antigen presenting cells which present an epitope comprising or consisting of a carbohydrate structure of interest, comprising the step of contacting antigen presenting cells with the glycosylated glycophorin peptide according to the first aspect of the invention or the conjugate according to the second aspect of the invention, wherein the glycosylated glycophorin peptide carries the carbohydrate structure of interest.

In a fourth aspect, the present invention provides an antigen presenting cell obtainable by the method according to the third aspect of the invention.

In a fifth aspect, the present invention provides a method for producing activated T cells against a carbohydrate structure of interest, comprising contacting T cells with antigen presenting cells according to the fourth aspect of the invention.

In a sixth aspect, the present invention provides an activated T cell obtainable by the method according to the fifth aspect of the invention.

According to a seventh aspect of the present invention, a medical use of the glycosylated glycophorin peptide according to the first aspect of the invention, the conjugate according to the second aspect of the invention, the antigen presenting cell according to the fourth aspect of the invention or the activated T cell according to the sixth aspect of the invention is provided.

Other objects, features, advantages and aspects of the present invention will become apparent to those skilled in the art from the following description and appended claims. It should be understood, however, that the following description, appended claims, and specific examples, which indicate preferred embodiments of the application, are given by way of illustration only. Various changes and modifications within the spirit and scope of the disclosed invention will become readily apparent to those skilled in the art from reading the following.

### DETAILED DESCRIPTION OF THE INVENTION

In a first aspect, the present invention provides a glycosylated glycophorin peptide comprising
(i) at least one amino acid independently selected from serine and threonine which carries a carbohydrate structure; and
(ii) at least 5 consecutive amino acids which are identical to or have at least 75% homology to an amino acid segment of the same length within the amino acid sequence of SEQ ID NO: 1.

The glycosylated glycophorin peptide in particular comprises at least 5, especially at least 6, at least 7, at least 8, at least 9, at least 10, at least 11, at least 12, at least 13, at least 14 or at least 15, preferably at least 8 or at least 9 consecutive amino acids which are identical to or have at least 75% homology, especially at least 80%, at least 85%, at least 90%, at least 95% or 100% homology to an amino acid segment of the same length within the amino acid sequence of SEQ ID NO: 1. In specific embodiments, the percentage homology in particular refers to the same percentage identity. In certain embodiments, the glycosylated glycophorin peptide is derived from the extracellular domain of glycophorin A. In these embodiments, the glycosylated glycophorin peptide comprises said consecutive amino acids which are identical or have a homology or identity as described above to an amino acid segment of the same length within positions 1 to 67 of the amino acid sequence of SEQ ID NO: 1. In particular, it comprises at least 5, especially at least 6, at least 7, at least 8, at least 9, at least 10, at least 11, at least 12, at least 13, at least 14 or at least 15, preferably at least 8 or at least 9 consecutive amino acids which are identical to or have at least 75% homology, especially at least 80%, at least 85%, at least 90%, at least 95% or 100% homology, or said percentage identity, to an amino acid segment of the same length within positions 1 to 67 of the amino acid sequence of SEQ ID NO: 1. In another embodiment, the glycosylated glycophorin peptide is derived from the transmembrane domain and/or the cytoplasmatic domain of glycophorin A. In these embodiments, the glycosylated glycophorin peptide comprises said consecutive amino acids which are identical or have a homology as described above to an amino acid segment of the same length within positions 60 to 131 of the amino acid sequence of SEQ ID NO: 1, in particular within positions 60 to 101 of the amino acid sequence of SEQ ID NO: 1 (transmembrane domain) or within positions 94 to 131 of the amino acid sequence of SEQ ID NO: 1 (cytoplasmatic domain).

In certain embodiments, glycosylated glycophorin peptide comprises from 7 to 30, especially from 8 to 25, from 10 to 23, preferably from 8 to 15 or from 15 to 23 consecutive amino acids which are identical to or have at least 75% homology, especially at least 80%, at least 85%, at least 90%, at least 95% or 100% homology to an amino acid segment of the same length within the amino acid sequence of SEQ ID NO: 1. In specific embodiments, the percentage homology in particular refers to the same percentage identity.

The remaining amino acids of the glycosylated glycophorin peptide may have any sequence, but are preferably also derived from glycophorin A. Hence, the entire amino acid sequence of the glycophorin peptide preferably is identical to or has at least 75%, especially at least 80%, at least 85%, at least 90%, at least 95% or 100% homology or at least 75%, especially at least 80%, at least 85%, at least 90%, at least 95% or 100% identity, to an amino acid segment of the same length within the amino acid sequence of SEQ ID NO: 1, in particular within positions 1 to 63, 60 to 101 or 94 to 131 of the amino acid sequence of SEQ ID NO: 1, preferably within positions 1 to 63 of the amino acid sequence of SEQ ID NO: 1.

In specific embodiments, the glycosylated glycophorin peptide comprises at least 5, especially at least 6, at least 7, at least 8, at least 9, at least 10, at least 11, at least 12, at least 13, at least 14 or at least 15, preferably at least 8 or at least 9 consecutive amino acids which are identical to an amino acid segment of the same length within the amino acid sequence of SEQ ID NO: 1, and wherein additionally the entire amino acid sequence of the glycosylated glycophorin peptide has at least 75%, especially at least 80%, at least 85%, at least 90%, at least 95% or 100% homology, or said percentage identity, to an amino acid segment of the same length within the amino acid sequence of SEQ ID NO: 1.

In certain embodiments, the consecutive amino acids having at least a specific percentage homology or identity to an amino acid segment of the same length within the amino acid sequence of SEQ ID NO: 1 or a specific part thereof, comprise 1 to 5, in particular 1 to 4 or 1 to 3, such as 1 or 2 amino acid substitutions, additions or deletions with respect to the amino acid segment of the same length within the amino acid sequence of SEQ ID NO: 1. Likewise, in certain embodiments, the entire amino acid sequence of the glycosylated glycophorin peptide having at least a specific percentage homology or identity to an amino acid segment of the same length within the amino acid sequence of SEQ ID NO: 1 or a specific part thereof, comprises 1 to 5, in particular 1 to 4 or 1 to 3, such as 1 or 2 amino acid substitutions, additions or deletions with respect to the amino acid segment of the same length within the amino acid sequence of SEQ ID NO: 1.

The amino acid sequences of SEQ ID NOs: 2 and 3 represent specific embodiments of the amino acid sequence of SEQ ID NO: 1. Any reference herein to the amino acid sequence of SEQ ID NO: 1 in particular also refers to the amino acid sequence of SEQ ID NO: 2 and/or the amino acid sequence of SEQ ID NO: 3. The amino acid sequence of SEQ ID NO: 2 represents human glycophorin type M and the amino acid sequence of SEQ ID NO: 3 represents human glycophorin type N.

The glycosylated glycophorin peptide may have a length of at least 5 amino acids, in particular at least 6, at least 7, at least 8, at least 9, at least 10, at least 11, at least 12, at least 13, at least 14 or at least 15 amino acids, preferably at least 8, more preferably at least 10, most preferably at least 12 amino acids. In particular, the glycosylated glycophorin peptide may have a length of 100 amino acids or less, such as 67 amino acids or less, 50 amino acids or less, 40 amino acids or less, 30 amino acids or less or 27 amino acids or less. In certain embodiments, the glycosylated glycophorin peptide has a length of 9 to 50 amino acids, in particular 10 to 40 amino acids, 12 to 30 amino acids, 13 to 28 amino acids, 14 to 26 amino acids or 15 to 24 amino acids. According to other embodiments, the glycosylated glycophorin peptide has a length of 5 to 15 amino acids, in particular 6 to 12 amino acids, 7 to 11 amino acids, 8 to 10 amino acids, or 8 or 9 amino acids.

In certain embodiments, the glycosylated glycophorin peptide has a length of from 15 to 24 amino acids and comprises at least 14 consecutive amino acids which are identical to an amino acid segment of the same length within positions 1 to 67 of the amino acid sequence of SEQ ID NO: 1, and wherein additionally the entire amino acid sequence of the glycosylated glycophorin peptide has at least 90% identity to an amino acid segment of the same length within positions 1 to 67 of the amino acid sequence of SEQ ID NO: 1.

In certain embodiments, the glycosylated glycophorin peptide comprises the amino acid sequence of position 34 to 42 of SEQ ID NO: 1 or an amino acid sequence which has at least 80%, in particular at 100% homology, especially said percentage identity, thereto. In further embodiments, the glycosylated glycophorin peptide comprises the amino acid sequence of position 48 to 55 of SEQ ID NO: 1 or an amino acid sequence which has at least 75%, in particular at least 85% or 100% homology, especially said percentage identity, thereto.

In particular embodiments, the glycosylated glycophorin peptide comprises or consists of an amino acid sequence selected from the group consisting of
(i) the amino acid sequence of position 1 to 9 of SEQ ID NO: 1, in particular SEQ ID NO: 2 or SEQ ID NO: 3;
(ii) the amino acid sequence of position 9 to 25 of SEQ ID NO: 1, in particular SEQ ID NO: 2;
(iii) the amino acid sequence of position 26 to 43 of SEQ ID NO: 1, in particular SEQ ID NO: 2;
(iv) the amino acid sequence of position 34 to 55 of SEQ ID NO: 1, in particular SEQ ID NO: 2;
(v) the amino acid sequence of position 41 to 55 of SEQ ID NO: 1, in particular SEQ ID NO: 2;
(vi) the amino acid sequence of position 45 to 55 of SEQ ID NO: 1, in particular SEQ ID NO: 2;
(vii) the amino acid sequence of position 48 to 55 of SEQ ID NO: 1, in particular SEQ ID NO: 2;
(viii) the amino acid sequence of position 48 to 63 of SEQ ID NO: 1, in particular SEQ ID NO: 2;
(ix) the amino acid sequence of position 21 to 40 of SEQ ID NO: 1, in particular SEQ ID NO: 2; and
(x) the amino acid sequence of position 21 to 45 of SEQ ID NO: 1, in particular SEQ ID NO: 2; and
(xi) an amino acid sequence which has at least 75%, in particular at least 80%, at least 85%, at least 90%, at least 95% or 100% homology, in particular said percentage identity, to one of the sequences according to (i) to (x).

In particular, the glycosylated glycophorin peptide comprises or consists of the amino acid sequence of position 26 to 43, positions 48 to 63, positions 34 to 55, positions 9 to 25 or positions 1 to 9 of SEQ ID NO: 1, in particular SEQ ID NO: 2 or SEQ ID NO: 3, or an amino acid sequence which has at least 85% identity thereto.

In specific embodiments, one or more threonine or serine residues within the amino acid sequence identical to or derived from the indicated part of SEQ ID NO: 1 carry a carbohydrate structure. In certain embodiments, in glycosylated glycophorin peptides which share a certain homology or identity with a specific amino acid sequence of SEQ ID NO: 1, the threonine or serine residue(s) carrying a carbohydrate structure is(are) also present in the amino acid sequence of SEQ ID NO: 1. In particular, the one or more threonine or serine residues carrying a carbohydrate structure correspond to serine at position 1 (Ser1), Ser2, threonine at position 3 (Thr3), Thr4, Thr10, Ser11, Thr12, Ser13, Ser14, Ser15, Thr17, Ser19, Ser22, Ser23, Thr25, Thr28, Thr33, Thr37, Ser44, Ser47, Ser49, Thr50, Ser52, Ser54, Thr58, Ser69, Thr74, Thr87, Ser92, Thr93, Ser102, Ser104, Ser111, Thr114, Ser119, Ser120, Thr128 and/or Ser129, respectively, of SEQ ID NO: 1. Especially, the one or more threonine or serine residues carrying a carbohydrate structure correspond to Ser2, Thr3, Thr4, Thr10, Ser11, Thr12, Ser13, Thr17, Ser22, Thr33, Thr37, Ser44, Ser47 or Thr50, respectively, of SEQ ID NO: 1. In certain embodiments, one of the residues carrying a carbohydrate structure corresponds to Thr33 of SEQ ID NO: 1 and/or one of the residues carrying a carbohydrate structure corresponds to Thr37 of SEQ ID NO: 1. In further embodiments, one of the residues carrying a carbohydrate structure corresponds to Thr50 of SEQ ID NO: 1. In even further embodiments, one of the residues carrying a carbohydrate structure corresponds to Ser2 of SEQ ID NO: 1, and/or one of the residues carrying a carbohydrate structure corresponds to Thr3 of SEQ ID NO: 1, and/or one of the residues carrying a carbohydrate structure corresponds to Thr4 of SEQ ID NO: 1.

In certain embodiments, the glycosylated glycophorin peptide comprises or consists of the amino acid sequence of position 26 to 43 of SEQ ID NO: 1, in particular SEQ ID NO: 2, wherein the threonine corresponding to position 33 of SEQ ID NO: 1 and/or the threonine corresponding to position 37 of SEQ ID NO: 1 carries a carbohydrate structure. In particular, the threonine corresponding to position 33 of SEQ ID NO: 1 or the threonine corresponding to position 37 of SEQ ID NO: 1 is the only amino acid in the glycosylated glycophorin peptide carrying a carbohydrate structure.

In other embodiments, the glycosylated glycophorin peptide comprises or consists of the amino acid sequence of position 48 to 63 of SEQ ID NO: 1, in particular SEQ ID NO: 2, wherein the threonine corresponding to position 50 of SEQ ID NO: 1 carries a carbohydrate structure. In particular, the threonine corresponding to position 50 of SEQ ID NO: 1 is the only amino acid in the glycosylated glycophorin peptide carrying a carbohydrate structure.

In further embodiments, the glycosylated glycophorin peptide comprises or consists of the amino acid sequence of position 1 to 9 of SEQ ID NO: 1, in particular SEQ ID NO: 2 or SEQ ID NO: 3, wherein the serine corresponding to position 2 of SEQ ID NO: 1 and/or the threonine corresponding to position 3 of SEQ ID NO: 1 and/or the threonine corresponding to position 4 of SEQ ID NO: 1 carries a carbohydrate structure. In particular, 1, 2 or all three of these residues carry a carbohydrate structure. In specific embodiments, no other amino acid residue of the glycosylated glycophorin peptide carries a carbohydrate structure.

In further embodiments, the glycosylated glycophorin peptide comprises or consists of the amino acid sequence of position 9 to 25 of SEQ ID NO: 1, in particular SEQ ID NO: 2, wherein the threonine corresponding to position 10 of SEQ ID NO: 1 and/or the serine corresponding to position 11 of SEQ ID NO: 1 and/or the threonine corresponding to position 12 of SEQ ID NO: 1 and/or the serine corresponding to position 13 of SEQ ID NO: 1 and/or the threonine corresponding to position 17 of SEQ ID NO: 1 and/or the serine corresponding to position 22 of SEQ ID NO: 1 carries a carbohydrate structure. In particular, 1, 2, 3, 4, 5 or all 6 of these residues carry a carbohydrate structure. In a certain embodiment, the amino acid residues corresponding to positions 12, 13, 17 and 22 of SEQ ID NO: 1 carry a carbohydrate structure. In a further embodiment, the amino acid residues corresponding to positions 10, 11, 12, 13, 17 and 22 of SEQ ID NO: 1 carry a carbohydrate structure. In specific embodiments, no other amino acid residue of the glycosylated glycophorin peptide carries a carbohydrate structure.

In further embodiments, the glycosylated glycophorin peptide comprises or consists of the amino acid sequence of position 34 to 55 of SEQ ID NO: 1, in particular SEQ ID NO: 2, wherein the threonine corresponding to position 37 of SEQ ID NO: 1 and/or the serine corresponding to position 44 of SEQ ID NO: 1 and/or the serine corresponding to position 47 of SEQ ID NO: 1 and/or the threonine corresponding to position 50 of SEQ ID NO: 1 carries a carbohydrate structure. In particular, 1, 2, 3 or all 4 of these residues, especially all 4 of these residues, carry a carbohydrate structure. In specific embodiments, no other amino acid residue of the glycosylated glycophorin peptide carries a carbohydrate structure.

In further embodiments, the glycosylated glycophorin peptide comprises or consists of the amino acid sequence of position 41 to 55 of SEQ ID NO: 1, in particular SEQ ID NO: 2, wherein the serine corresponding to position 44 of SEQ ID NO: 1 and/or the serine corresponding to position 47 of SEQ ID NO: 1 and/or the threonine corresponding to position 50 of SEQ ID NO: 1 carries a carbohydrate structure. In particular, 1, 2 or all 3 of these residues carry a carbohydrate structure. In specific embodiments, no other amino acid residue of the glycosylated glycophorin peptide carries a carbohydrate structure.

In further embodiments, the glycosylated glycophorin peptide comprises or consists of the amino acid sequence of position 45 to 55 of SEQ ID NO: 1, in particular SEQ ID NO: 2, wherein the serine corresponding to position 47 of SEQ ID NO: 1 and/or the threonine corresponding to position 50 of SEQ ID NO: 1 carries a carbohydrate structure. In particular, either one or both of these residues carry a carbohydrate structure. In specific embodiments, no other amino acid residue of the glycosylated glycophorin peptide carries a carbohydrate structure.

In further embodiments, the glycosylated glycophorin peptide comprises or consists of the amino acid sequence of position 48 to 55 of SEQ ID NO: 1, in particular SEQ ID NO: 2, wherein the threonine corresponding to position 50 of SEQ ID NO: 1 carries a carbohydrate structure. In specific embodiments, no other amino acid residue of the glycosylated glycophorin peptide carries a carbohydrate structure.

In certain embodiments, the glycosylated glycophorin peptide comprises or consists of an amino acid sequence selected from the group consisting of
(i) the amino acid sequence of position 55 to 64 of SEQ ID NO: 1, in particular SEQ ID NO: 2;
(ii) the amino acid sequence of position 58 to 72 of SEQ ID NO: 1, in particular SEQ ID NO: 2;
(iii) the amino acid sequence of position 69 to 77 of SEQ ID NO: 1, in particular SEQ ID NO: 2;
(iv) the amino acid sequence of position 75 to 89 of SEQ ID NO: 1, in particular SEQ ID NO: 2;
(v) the amino acid sequence of position 89 to 98 of SEQ ID NO: 1, in particular SEQ ID NO: 2;
(vi) the amino acid sequence of position 92 to 106 of SEQ ID NO: 1, in particular SEQ ID NO: 2;
(vii) an amino acid sequence which has at least 75%, in particular at least 80%, at least 85%, at least 90%, at least 95% or 100% homology, in particular said percentage identity, to one of the sequences according to (i) to (vi).

In specific embodiments, the glycosylated glycophorin peptide is capable of binding to MHC class II proteins, in particular human MHC class II proteins such as those encoded by a HLA-DR gene. In these embodiments, the glycosylated glycophorin peptide preferably has a length of 9 to 50 amino acids, more preferably 12 to 30 amino acids, most preferably 15 to 24 amino acids.

In further embodiments, the glycosylated glycophorin peptide is capable of binding to MHC class I proteins. In these embodiments, the glycosylated glycophorin peptide preferably has a length of 5 to 14 amino acids, in particular 8 to 11 amino acids, such as 8 or 9 amino acids.

The amino acid residue(s) carrying a carbohydrate structure may be part of the MHC binding motif or may be positioned outside of the MHC binding motif of the glycosylated glycophorin peptide. In certain embodiments, at least one of the amino acids carrying a carbohydrate structure is part of the MHC binding motif. In particular, all amino acids carrying a carbohydrate structure are part of the MHC binding motif. In other embodiments, the amino acids carrying a carbohydrate structure are not part of the MHC binding motif. In further embodiments, the glycosylated glycophorin peptide comprises one or more amino acid residues of the MHC binding motif which carry a carbohydrate structure as well as one or more amino acid residues outside of the MHC binding motif which carry a carbohydrate structure. In certain embodiments the MHC binding motif is an MHC class II binding motif or an MHC class I binding motif, in particular an MHC class II binding motif.

The glycosylated glycophorin peptide comprises at least one amino acid, in particular at least two amino acids, especially one or two amino acids, independently selected from serine and threonine, which carry a carbohydrate structure. In certain embodiments, the amino acid(s) carrying a carbohydrate structure is(are) threonine.

In certain embodiments, the carbohydrate structure of the glycosylated glycophorin peptide is a carbohydrate tumor epitope (also referred to as tumor-associated carbohydrate antigen). A carbohydrate tumor epitope in particular is a carbohydrate structure which is present on the surface of tumor cells. In particular, the carbohydrate tumor epitope is not or to a lesser amount present or not or to a lesser amount accessible on healthy cells of a human or animal being. A carbohydrate tumor epitope is not accessible, for example, if it is present on the basolateral membrane of a polarized cell, but not on the apical membrane, or if it is masked by other structures which prevent binding of antibodies or receptors to said structure.

The carbohydrate structure may be selected from the group consisting of TFα, Tn, sialyl-Tn, sialyl-TF, Globo-H, Lewis-Y, sialyl-Lewis-A, sialyl-Lewis-X, polysialic acid, Lewis-X, GM2, GD2, GD3, 9-O-acetyl GD3, GD3L, fucosyl GM1, Lewis-A, Lewis-B, sLac, sialylated type 1 chain, CA 19-9 antigen, CA 72-4 antigen and CA-50 antigen. In particular embodiments, the carbohydrate structure has a formula selected from the group consisting of
(i) Galβ1,3-GalNAcα1- (TFα);
(ii) GalNAcα1- (Tn);
(iii) Siaα2,3-Galβ1,3-GalNAcα1- (sialyl-TFα);
(iv) Siaα2,6-GalNAcα1- (sialyl-Tn);
(v) Galβ1,3-(Fucα1,4)-GlcNAcβ1- (Lewis-A);
(vi) Siaα2,3-Galβ1,3-(Fucα1,4)-GlcNAcβ1- (sialyl-Lewis-A);
(vii) Galβ1,4-(Fucα1,3)-GlcNAcβ1- (Lewis-X);
(viii) Siaα2,3-Galβ1,4-(Fucα1,3)-GlcNAcβ1- (sialyl-Lewis-X);
(ix) Fucα1,2-Galβ1,4-(Fucα1,3)-GlcNAcβ1- (Lewis-Y);
(x) Fucα1,2-Galβ1,3-(Fucα1,4)-GlcNAcβ1- (Lewis-B); and
(xi) Fucα1,2-Galβ1,3-GalNAcβ1,3-Galα1,4-Galβ1,4-Glcβ1- (Globo-H).

In the indicated carbohydrate structures, Gal represents galactose, GalNAc represents N-acetyl galactosamine, Glc represents glucose, GlcNAc represents N-acetyl glucosamine, Fuc represents fucose, and Sia represents sialic acid, in particular N-acetyl neuraminic acid.

If the glycosylated glycophorin peptide comprises more than one amino acid carrying a carbohydrate structure, the carbohydrate structures may be the same or different and may be independently selected from the above examples. In certain embodiments, all carbohydrate structures of the glycosylated glycophorin peptide are the same. In specific embodiments, the carbohydrate structure has the formula Galβ1,3-GalNAcα1-(TFα). In further embodiments, the carbohydrate structure has the formula GalNAcα1-(Tn).

In a second aspect, the present invention provides a conjugate comprising the glycosylated glycophorin peptide according to the first aspect of the invention covalently coupled to another agent. The other agent may be any agent, in particular an agent which supports the desired function of the glycosylated glycophorin peptide. The other agent may for example enhance the stability of the glycosylated glycophorin peptide, enhance its serum half-life in the human circulation, enhance its uptake by antigen-presenting cells, reduce its degradation by proteases and/or peptidases, and/or enhance its solubility. The conjugate may comprise more than one other agent, wherein the two or more other agents may be the same or different. The other agent in particular is covalently coupled to the glycosylated glycophorin peptide. In certain embodiments, the other agent is a peptide, protein or lipid. Certain examples of suitable other agents being a protein or peptide are tetanus toxoid, diphtheria toxoid, ovalbumin, diphtheria CRM197, bovine serum albumin, keyhole limpet hemocyanin, N. meningitis outer membrane protein, nontypeable Haemophilus influenzae protein D and human immunodeficiency virus transactivating regulatory protein (HIV TAT), as well as peptides derived from these proteins, in particular HIV TAT peptides. Suitable lipids for use a the other agent in the conjugate are for example S-[2,3-bis(palmitoyloxy)propyl]cysteine (Pam2Cys), N-palmitoyl-S-[2,3-bis(palmitoyloxy)-(2RS)-propyl]-(R)-cysteine (Pam3Cys), monophosphoryl lipid A, lipopolysaccharide (LPS) and LPS derivatives. The other agent may also be a complex structure such as a liposome or a virus-like particle. In certain embodiments, the conjugate is a fusion protein. The conjugate, however, is not the full length human glycophorin A having the amino acid sequence of SEQ ID NO: 1, or another naturally occurring glycophorin protein. In particular, the other agent does not share an amino acid sequence identity over its entire length of more than 50% with the amino acid sequence of SEQ ID NO: 1.

In a third aspect, the present invention provides a method for producing antigen presenting cells which present an epitope comprising or consisting of a carbohydrate structure of interest, comprising the step of contacting antigen presenting cells with the glycosylated glycophorin peptide according to the first aspect of the invention or the conjugate according to the second aspect of the invention, wherein the glycosylated glycophorin peptide carries the carbohydrate structure of interest.

In particular, the method for producing antigen presenting cells which present an epitope comprising or consisting of a carbohydrate structure of interest comprises one or more, in particular all of the following steps:
(a) providing immature antigen presenting cells;
(b) maturing the immature antigen presenting cells, for example by cultivation in a cell culture medium supplemented with tumor necrosis factor α (TNF-α), in particular at a concentration of about 10 ng/ml to about 1 μg/ml, such as 75 ng/ml;
(c) contacting the mature antigen presenting cells with the glycosylated glycophorin peptide according to the first aspect of the invention or the conjugate according to the second aspect of the invention, carrying the carbohydrate structure of interest, in particular on day one or two of the maturation, wherein the concentration of the glycosylated glycophorin peptide or conjugate in the cell culture medium is in the range of from about 1 μg/ml to about 1 mg/ml, in particular from about 2 μg/ml to about 100 μg/ml, such as about 10 μg/ml;
(d) incubating the antigen presenting cells in the presence of the glycosylated glycophorin peptide or conjugate until at least a part of the antigen presenting cells is loaded with the glycosylated glycophorin peptide or conjugate, in particular using an incubation time of several hours to two days; and
(e) harvesting the loaded antigen presenting cells.

In certain embodiments, the antigen presenting cells are dendritic cells. Furthermore, in specific embodiments the antigen presenting cells are human cells such as human dendritic cells. In certain embodiments, the carbohydrate structure of interest has the formula Galβ1,3-GalNAcα1-.

In particular, the glycosylated glycophorin peptide according to the first aspect of the invention is capable of uptake by antigen presenting cells and presentation of at least a part thereof on the surface of the antigen presenting cells, wherein the part thereof comprises at least one amino acid residue carrying a carbohydrate structure.

In a fourth aspect, the present invention provides an antigen presenting cell obtainable by the method according to the third aspect of the invention. The antigen presenting cell presents on its surface the glycosylated glycophorin peptide according to the first aspect of the invention, or a fragment thereof which comprises at least one amino acid carrying the carbohydrate structure of interest. In certain embodiments, the antigen presenting cell is a human dendritic cell and the carbohydrate structure of interest has the formula Galβ1,3-GalNAcα1-.

In a fifth aspect, the present invention provides a method for producing activated T cells against a carbohydrate structure of interest, comprising contacting T cells with antigen presenting cells according to the fourth aspect of the invention.

The antigen presenting cells according to the fourth aspect of the invention may be used for priming the T cells or for restimulating the T cells after priming with antigen presenting cells loaded with a different agent carrying the carbohydrate structure of interest. In particular, the method comprises one or more, especially all of the following steps:
(a) priming immature T cells with antigen presenting cells loaded with a first agent carrying the carbohydrate structure of interest;
(b) incubating the T cells for proliferation, in particular for about 4 to 28 days, such as for about 8 to 17 days;
(c) restimulating the T cells with antigen presenting cells loaded with a second agent carrying the carbohydrate structure of interest;
   wherein either the first agent or the second agent is the glycosylated glycophorin peptide according to the first aspect of the invention or the conjugate according to the second aspect of the invention; and wherein the other agent is a different agent carrying the carbohydrate structure of interest. The other agent may for example be a microorganism such as AG6, MU1 and Coreotics, or a cell line such as NM-F9 and NM-D4.

AG6 was deposited on Oct. 20, 2006 under the accession number DSM 18726 according to the requirements of the Budapest Treaty at the Deutsche Sammlung von Mikroorganismen und Zellkulturen (DSMZ), Inhoffenstraβe 7B, 38124 Braunschweig (DE) by Glycotope GmbH, Robert-Rössle-Str. 10, 13125 Berlin (DE).

MU1 was deposited on Oct. 20, 2006 under the accession number DSM 18728 according to the requirements of the Budapest Treaty at the Deutsche Sammlung von Mikroorganismen und Zellkulturen (DSMZ), Inhoffenstraβe 7B, 38124 Braunschweig (DE) by Glycotope GmbH, Robert-Rössle-Str. 10, 13125 Berlin (DE).

Coreotics was deposited on Jul. 12, 2011 under the accession number DSM 25004 according to the requirements of the Budapest Treaty at the Deutsche Sammlung von Mikroorganismen und Zellkulturen (DSMZ), Inhoffenstraβe 7B, 38124 Braunschweig (DE) by Glycotope GmbH, Robert-Rössle-Str. 10, 13125 Berlin (DE).

NM-F9 was deposited on Aug. 14, 2003 under the accession number DSM ACC2606 according to the requirements of the Budapest Treaty at the Deutsche Sammlung von Mikroorganismen und Zellkulturen (DSMZ), Mascheroder Weg 1b, 38124 Braunschweig (DE) by Nemod Biotherapeutics GmbH & Co. KG, Robert-Rössle-Str. 10, 13125 Berlin (DE). The Applicant is entitled to refer to this biological material since it was in the meantime assigned from Nemod Biotherapeutics GmbH & Co. KG to Glycotope GmbH.

NM-D4 was deposited on Aug. 14, 2003 under the accession number DSM ACC2605 according to the requirements of the Budapest Treaty at the Deutsche Sammlung von Mikroorganismen und Zellkulturen (DSMZ), Mascheroder Weg 1 b, 38124 Braunschweig (DE) by Nemod Biotherapeutics GmbH & Co. KG, Robert-Rössle-Str. 10, 13125 Berlin (DE). The Applicant is entitled to refer to this biological material since it was in the meantime assigned from Nemod Biotherapeutics GmbH & Co. KG to Glycotope GmbH.

Incubation and cultivation of the antigen presenting cells and T cells is in particular performed at standard conditions for human cells, such as for example at 37° C., 3% to 10% CO₂ and 80% to 98% relative humidity.

In a sixth aspect, the present invention provides an activated T cell obtainable by the method according to the fifth aspect of the invention. The activated T cell is directed against the carbohydrate epitope of interest.

According to a seventh aspect of the present invention, a medical use of the glycosylated glycophorin peptide according to the first aspect of the invention, the conjugate according to the second aspect of the invention, the antigen presenting cell according to the fourth aspect of the invention or the activated T cell according to the sixth aspect of the invention is provided.

The use in medicine in particular includes the treatment and/or prevention of cancer which is positive for the carbohydrate structure of the glycosylated glycophorin peptide. In certain embodiments, the use in medicine is vaccination against such cancer. In particular, the cancer is positive for TFα and the glycosylated glycophorin peptide carries a carbohydrate structure being TFα. In specific embodiments, the cancer is selected from the group consisting of cancer of the bile duct, breast cancer, colon cancer, kidney cancer, liver cancer, lung cancer, ovary cancer, cervical cancer, prostate cancer, skin cancer, gastric cancer, pancreatic cancer, small intestine cancer, leukemia such as chronic lymphocytic leukemia and chronic myelogenous leukemia, lymphoma such as Burkitt's lymphoma, multiple myeloma, and cancer of the uterus, and/or metastases derived from any of these cancers. In certain embodiments, the medical use is for treating a human patient.

Furthermore, the invention provides a pharmaceutical composition, in particular a vaccine, comprising the glycosylated glycophorin peptide according to the first aspect of the invention, the conjugate according to the second aspect of the invention, the antigen presenting cell according to the fourth aspect of the invention or the activated T cell according to the sixth aspect of the invention. The pharmaceutical composition may further comprise an adjuvant.

### DEFINITIONS

As used herein, the following expressions are generally intended to preferably have the meanings as set forth below, except to the extent that the context in which they are used indicates otherwise.

The expression “comprise”, as used herein, besides its literal meaning also includes and specifically refers to the expressions “consist essentially of” and “consist of”. Thus, the expression “comprise” refers to embodiments wherein the subject-matter which “comprises” specifically listed elements does not comprise further elements as well as embodiments wherein the subject-matter which “comprises” specifically listed elements may and/or indeed does encompass further elements. Likewise, the expression “have” is to be understood as the expression “comprise”, also including and specifically referring to the expressions “consist essentially of” and “consist of”.

As used herein, the term “peptide” refers to a molecular chain of amino acids. A peptide can contain any of the naturally occurring amino acids as well as artificial amino acids and can be of biologic or synthetic origin. A peptide may be modified, naturally (post-translational modifications) or synthetically, by e.g. glycosylation, amidation, carboxylation and/or phosphorylation. A peptide comprises at least two amino acids, but does not have to be of any specific length; this term does not include any size restrictions. Preferably, a peptide comprises at least 5 amino acids, preferably at least 8 amino acids, and not more than 100 amino acids, preferably not more than 67 amino acids, not more than 50 amino acids or not more than 30 amino acids.

A “homology” of an amino acid sequence to a reference sequence is determined over the entire length of the reference sequence. Groups of amino acids which are considered homologous with each other are known in the art. Amino acid sequence homology can be determined, for example, using the “BLAST” internet homepage of the National Center for Biotechnology Information (NCBI) (http://blast.ncbi.nlm.nih.gov) and the standard parameters of the “blastp” program. In preferred embodiments, “homology” in particular refers to identity of the amino acids. An amino acid sequence having a certain percentage, e.g. at least 75%, homology to a reference amino acid sequence in particular has said percentage, e.g. at least 75%, identity to said reference amino acid sequence.

The term “cancer” according to the invention in particular comprises leukemias, seminomas, melanomas, teratomas, lymphomas, neuroblastomas, gliomas, rectal cancer, endometrial cancer, kidney cancer, adrenal cancer, thyroid cancer, blood cancer, skin cancer, cancer of the brain, cancer of the urogenital or gynecological system, cervical cancer, intestinal cancer, liver cancer, colon cancer, stomach cancer, intestine cancer, head and neck cancer, gastrointestinal cancer, lymph node cancer, cancer of the endocrine system, esophagus cancer, colorectal cancer, pancreas cancer, ear, nose and throat (ENT) cancer, bone cancer, breast cancer, prostate cancer, cancer of the uterus, ovarian cancer and lung cancer and the metastases thereof. Examples thereof are lung carcinomas, colorectal carcinomas, head and neck carcinomas, or metastases of the cancer types or tumors described above. The term cancer according to the invention also comprises cancer metastases.

The term “pharmaceutical composition” particularly refers to a composition suitable for administering to a human or animal, i.e., a composition containing components which are pharmaceutically acceptable. Preferably, a pharmaceutical composition comprises an active compound or a salt or prodrug thereof together with a carrier, diluent or pharmaceutical excipient such as buffer, preservative and tonicity modifier.

### FIGURES

FIG. 1 shows restimulation of TFα-specific T cells with aGPA. PBMCs of a healthy donor were twice primed with DCs loaded with a TFα positive B. ovatus strain (BO+). Restimulation with DCs loaded with lysate of a TFα positive (HC+) or TFα negative (HC−) human cell line was analyzed by IFN-γ ELISPOT. T cells restimulated with TFα positive cells were further cultivated and restimulated in a second round with aGPA- and GPA-loaded DCs, which was analyzed by IFN-γ ELISPOT. The mean numbers of IFN-γ+spots+SEM of one experiment performed in triplicates (1^{st} restimulation) or of four replicates (2^{nd} restimulation) are shown. IFN-γ+spots of PBMCs without restimulation were subtracted. Statistical analysis was performed using 1 way ANOVA (***: P<0.001).

FIG. 2 shows presentation of TFα on DCs after loading with aGPA. GPA- and aGPA-loaded cells were stained with Nemod-TF1 and Nemod-TF2 and analyzed by flow cytometry. The subpopulations of CD80+CD209+, CD80+CD209− and CD80−CD209− cells were analyzed separately. The mean percentages of positive cells+SD of one experiment performed in duplicates are shown.

FIG. 3 shows testing of direct attachment of TFα from aGPA to DCs. DCs were loaded with aGPA and GPA for indicated time periods. Presentation of TFα was analyzed on the subpopulations of CD80+CD209+ (a), CD80+CD209− (b) and CD80−CD209− (c) DCs by flow cytometry using Nemod-TF1. Data are presented as mean percentage of positive cells±SD of one experiment performed in duplicates.

FIG. 4 shows effects of antigen processing inhibitors on presentation of TFα derived from aGPA. DCs were pretreated with indicated concentrations of chloroquine (a) and ammonium chloride (b) before aGPA was added. Presentation of TFα was analyzed by flow cytometry using Nemod-TF1. Data are presented as the percentage of the relative presentation which was calculated according to the following formula: (percentage of positive cells incubated with antigen processing inhibitors)/(percentage of positive cells of untreated cells)×(100)). The means of one experiment performed in duplicates are shown.

FIG. 5 shows analysis of aGPA-loaded DCs with GPA-specific antibodies. Unloaded DCs and DCs loaded with aGPA and GPA were stained with a set of GPA-specific antibodies. Nemod-TF1 and B/A11 served as positive and negative controls, respectively. Binding was analyzed by flow cytometry on CD80+CD209− DCs. The mean percentages of positive cells+SD of one experiment performed in duplicates are shown. Values ≦0 were defined as not detectable (n.d.).

FIG. 6 shows analysis of DCs loaded with TFα-glycosylated GPA-derived peptides. DCs were loaded with the biotin-conjugated peptides GPA1-9(3TF), GPA9-25(4TF), GPA9-25(6TF), GPA26-43(2TF), GPA34-55(4TF), GPA41-55(3TF), GPA45-55(2TF), GPA48-55(1TF) and GPA48-63(1TF). Presentation was determined by fluorescence-labeled streptavidin. The subpopulations of CD80+CD209+, CD80+CD209− and CD80−CD209− mature DCs were analyzed separately. Mean percentages+SD of one experiment performed in duplicates are shown.

FIG. 7 shows analysis of GPA26-43(2TF) peptide-loaded DCs with a GPA-specific antibody. DCs were loaded with GPA26-43(2TF). Unloaded DCs and DCs loaded with aGPA and GPA, respectively, served as controls. Cells were stained with the GPA-specific antibody A83-C/B12 and analyzed by flow cytometry. The mean percentages+SD of one experiment performed in duplicates are shown.

FIG. 8 shows priming with a TFα positive B. ovatus strain and restimulation with TFα-glycosylated GPA-derived peptides. PBMCs of a healthy donor were primed with DCs loaded with TFα positive B. ovatus (BO+). Restimulation with GPA26-43(2TF) and GPA34-55(4TF) was analyzed by IFN-γ ELISPOT. The mean numbers of IFN-γ+spots+SEM of one experiment performed in triplicates is shown.

FIG. 9 shows analysis of DCs loaded with different variants of GPA26-43. DCs were loaded with the biotin-conjugated peptides. Presentation was determined by fluorescence-labeled streptavidin. The subpopulations CD80+CD209+, CD80+CD209- and CD80−CD209− mature DCs were analyzed separately. Mean percentages+SD of one experiment performed in triplicates are shown.

FIG. 10 shows priming of T cells with DCs loaded with TFα glycosylated GPA-derived peptides. PBMCs of a healthy donor were primed with DCs loaded with the TFα glycosylated GPA-derived peptides GPA9-25(4TF), GPA9-25(6TF) or GPA34-55(4TF). Restimulation with DCs loaded with the same peptide, lysates of TF-positive human cells or aGPA was analyzed by IFN-γ ELISPOT. No restimulation and restimulation with DCs loaded with TF-negative cell lysates or GPA were used as controls. Results are shown as relative IFN-γ secretion.

### EXAMPLES

### Example 1

### Dendritic Cells Loaded with aGPA Activate TFα-Specific T Cells

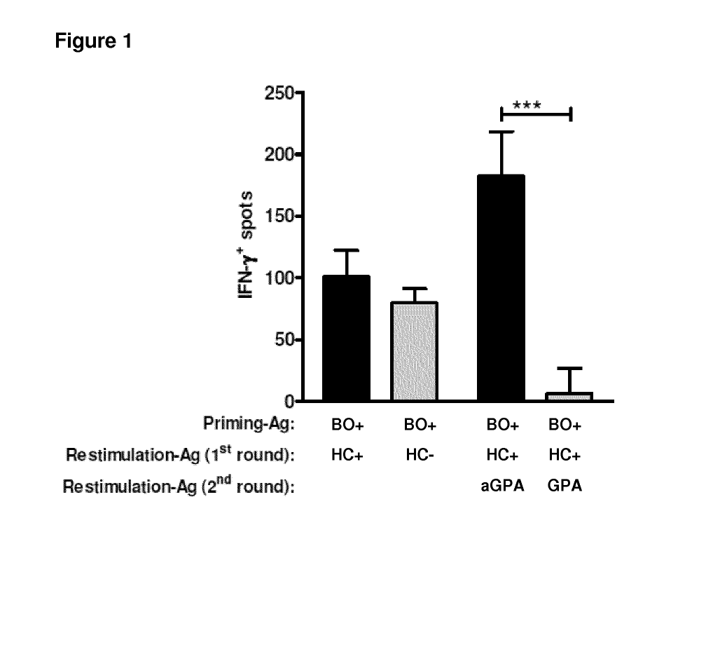

In order to analyze whether asialoglycophorin A (aGPA)-loaded dendritic cells (DCs) have the potential to activate TFα-specific T cells, a T cell culture, which was primed with a TFα positive B. ovatus strain and restimulated with lysate of a TFα positive human cell line, was restimulated in a second round with dendritic cells loaded with either aGPA or glycophorin A (GPA). Restimulation with aGPA-loaded dendritic cells resulted in a visible activation of T cells, whereas GPA-loaded dendritic cells showed almost no restimulation (FIG. 1), indicating that aGPA (but not GPA) loaded on dendritic cells is recognized by TFα-specific T cells.

### Example 2

### TFα Derived from aGPA is Presented on Dendritic Cells

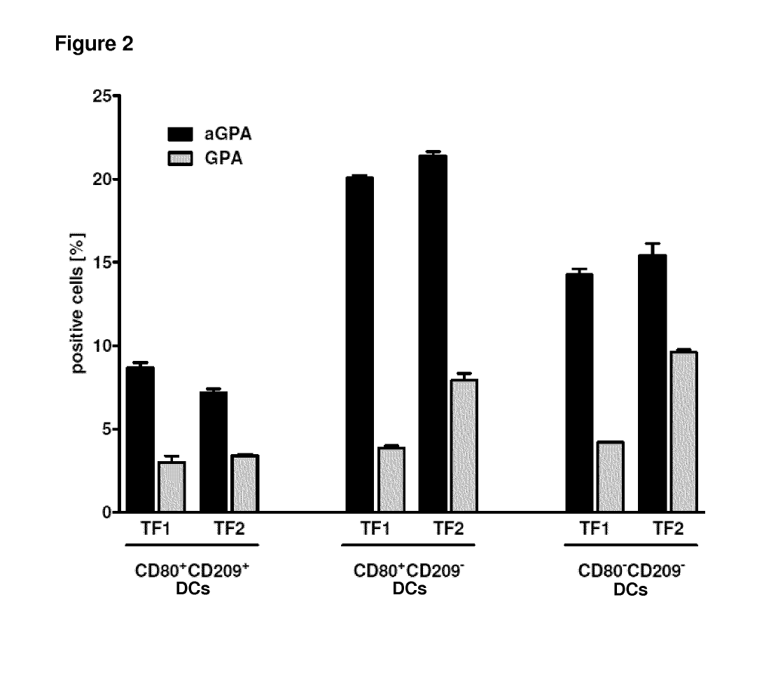

In order to analyze the antigen presentation of aGPA in more detail, dendritic cells loaded with aGPA and GPA were analyzed by flow cytometry using anti-TFα antibodies and for co-staining anti-CD80 and anti-CD209 antibodies. DCs loaded with aGPA showed an increased percentage of Nemod-TF1- and Nemod-TF2-positive cells compared to GPA-loaded cells in all three subpopulations (CD80+CD209+, CD80+CD209− and CD80−CD209− DCs) (FIG. 2). These results indicate that DCs loaded with aGPA present TF-α glycosylated peptides on their surface.

### Example 3

### TFα Derived from aGPA does not Bind Unspecifically to DCs

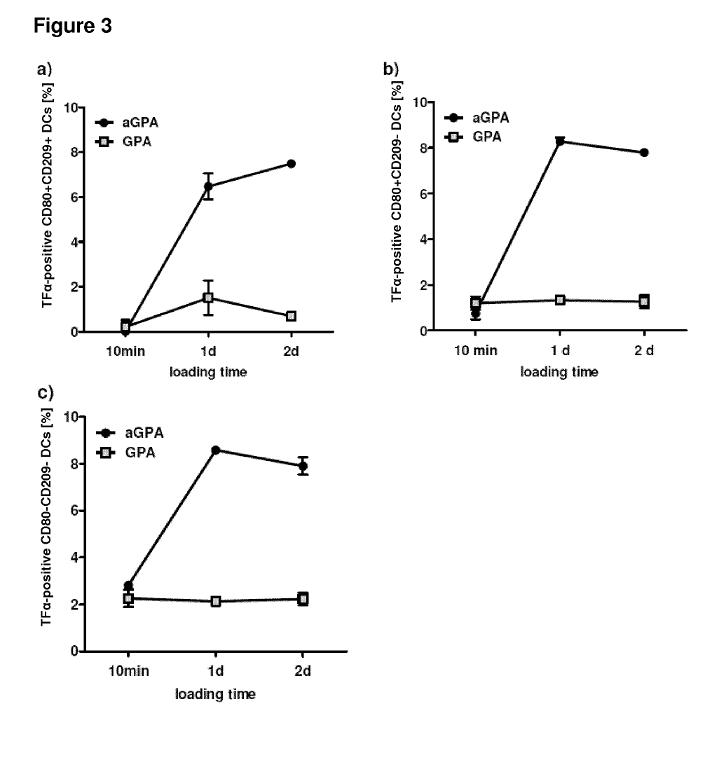

In order to test whether aGPA is unspecifically attached to the cell surface of DCs, aGPA was added 10 min prior to the analysis of surface presentation with the TFα-specific antibody Nemod-TF1 and compared to TFα-presentation after loading times of 1 and 2 d. In all three subpopulations of DCs (CD80+CD209+, CD80+CD209- and CD80−CD209−) no presentation was found when aGPA was added just before staining, indicating that in all subpopulations aGPA was processed and presented on MHC proteins, and did not just attach unspecifically to the cell surface (FIG. 3). This is important since the presentation on MHC proteins is crucial for activation of TF-α specific T cells.

### Example 4

### TFα Derived from aGPA is Processed by the Cell

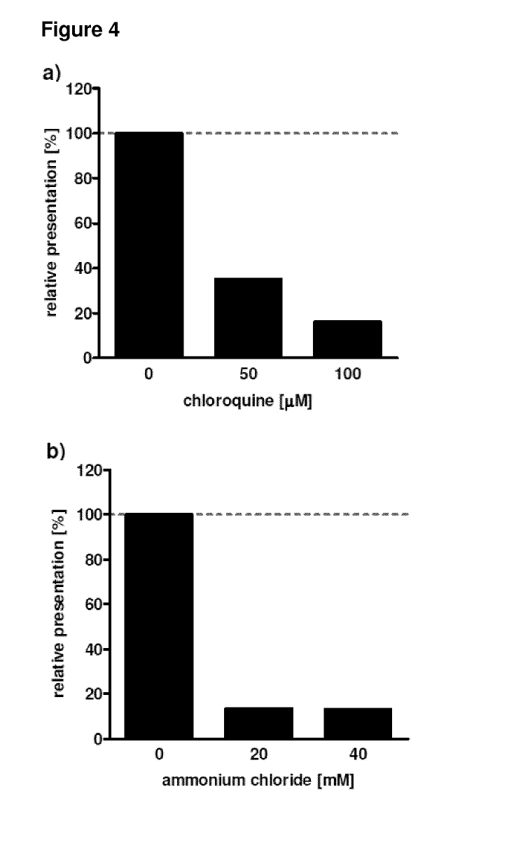

In order to characterize the processing pathway of aGPA, dendritic cells were treated with inhibitors of lysosomal acidification, chloroquine and ammonium chloride (MHC class II processing) prior to loading. Inhibition of the MHC class II pathway with chloroquine and ammonium chloride blocked the presentation of TFα (FIG. 4). These findings verify that aGPA is processed in the cell before TFα is presented on the cell surface. Furthermore, they demonstrate that TFα derived from aGPA is processed by the MHC class II pathway, and indicate that TFα-glycosylated peptides are presented via MHC class II molecules to T cells.

### Example 5

### Dendritic Cells Loaded with aGPA Present TFα-Glycosylated Peptides

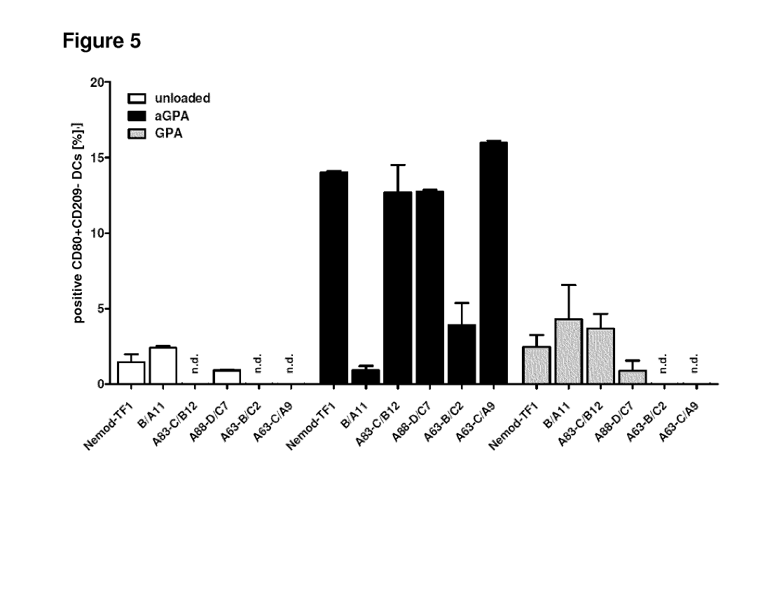

A set of antibodies against GPA was used to determine the peptides of GPA presented by dendritic cells. The antibodies A88-D/C7 and A63-B/C2 recognize sequence epitopes within aa36-45 and aa46-55 of GPA, respectively. A83-C/B12 binds to a conformational epitope within the sequence aa21-40 of GPA. The exact epitope of A63-C/A9 is unclear, but it is located in the extracellular domain. All antibodies bind better to aGPA than to GPA and are therefore suitable for detection of TFα-glycosylated peptides. Dendritic cells were loaded with aGPA and analyzed with the GPA-specific antibodies. Unloaded DCs and DCs loaded with GPA served as controls. Among the four antibodies directed towards GPA, three antibodies, A83-C/B12, A88-D/C7 and A63-C/A9, stained aGPA-loaded CD80+CD209− DCs comparable to Nemod-TF1, whereas on unloaded or GPA-loaded cells they showed only weak or no binding (FIG. 5). Binding of A83-C/B12 and A88-D/C7 indicates that the sequence aa21-45 is at least partly presented. Furthermore, binding of all three antibodies clearly demonstrates that the presented sequences were TFα-glycosylated. Interestingly, the antibody A63-B/C2 stained a lower percentage of aGPA-loaded CD80+CD209+ mature DCs than the other GPA-specific antibodies. The reduced staining of A63-B/C2 suggests that either during processing of aGPA its epitope was destroyed or the respective sequence, aa46-55, was not accessible for the antibody, possibly because it was bound in the peptide-binding groove of the MHC molecule. Similar binding patterns of the antibodies were seen for the subpopulations of CD80+CD209+ and CD80−CD209− DCs (data not shown).

### Example 6

### GPA-Derived TF-α Glycosylated Peptides are Presented on DCs

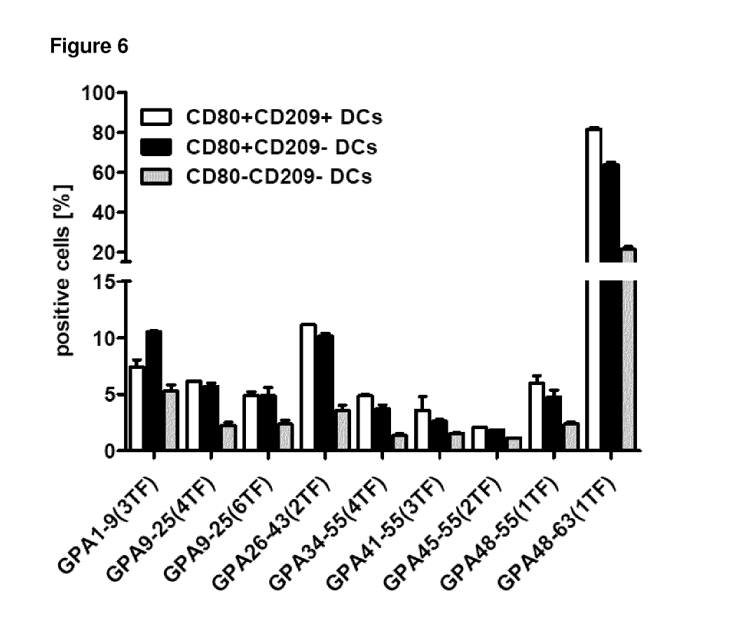

Since it was found that DCs loaded with aGPA present TFα-glycosylated peptides, it was of interest whether DCs can directly be loaded with GPA-derived TF-α glycosylated peptides. For that reason the GPA-derived TF-α glycosylated peptides GPA1-9(3TF), GPA9-25(4TF), GPA9-25(6TF), GPA26-43(2TF), GPA34-55(4TF), GPA41-55(3TF), GPA45-55(2TF), GPA48-55(1TF) and GPA48-63(1TF) were employed. Interestingly, all used TF-α glycosylated peptides were presented on DCs, whereas the percentage of positive cells varied between the peptides (FIG. 6). In summary, the TF-α glycosylated peptides GPA1-9(3TF), GPA26-43(2TF) and GPA48-63(1TF) showed strongest presentation on DCs.

### Example 7

### Presentation of GPA26-43(2TF) on DCs can be Detected by the GPA-Specific Antibody A83-C/B12

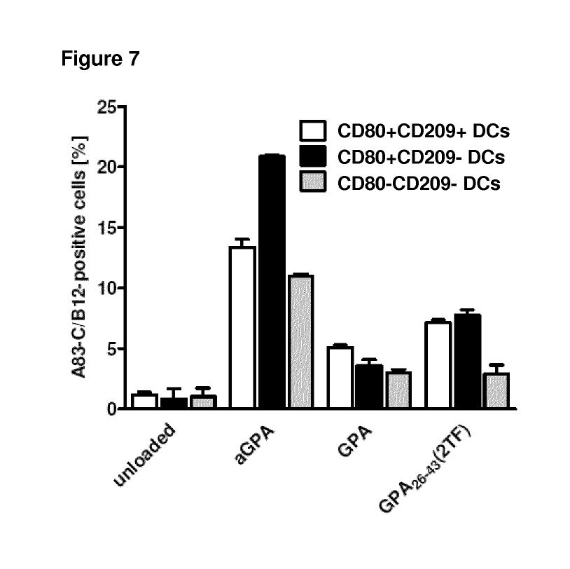

In addition to the analysis of peptide presentation via fluorescence-labeled streptavidin, it was tested whether the presentation of the peptide GPA26-43(2TF) can be detected using the GPA-specific antibody A83-C/B12. Unloaded cells and cells loaded with aGPA and GPA, respectively, were used as controls. Staining with the antibody A83-C/B12 clearly confirmed that GPA26-43(2TF) was presented on DCs (FIG. 7). In summary, GPA26-43(2TF) was identified as a TFα-glycosylated peptide which is presented on DCs.

### Example 8

### GPA-Derived TF-α Glycosylated Peptides can Activate TFα-Specific T Cells

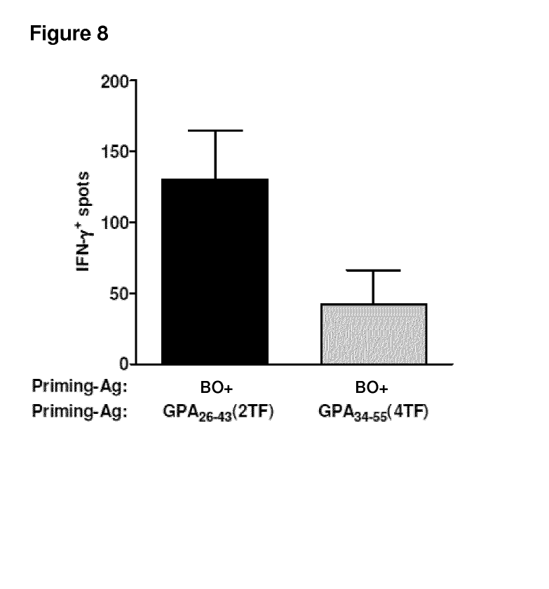

In order to analyze whether DCs loaded with peptide GPA26-43(2TF) or GPA34-55(4TF) can activate TFα-specific T cells, they were used for restimulation of T cells primed with a TFα-positive B. ovatus strain. Restimulation was detected by an IFN-γ ELISPOT assay. Restimulation with GPA26-43(2TF)-loaded DCs resulted in an stronger activation of T cells compared to DCs loaded with GPA34-55(4TF), consistent with the finding that GPA26-43(2TF) showed better presentation on DCs than GPA34-55(4TF) (FIG. 8). Since TFα is the only overlapping structure between the TFα-positive B. ovatus strain and the TFα-glycosylated GPA-derived peptides GPA26-43(2TF) and GPA34-55(4TF), it is obvious that the restimulated T cells were TFα-specific. The results demonstrate that glycosylated glycophorin peptides are capable of inducing a cellular immune response against their carbohydrate structure. This immune response, when elicited in a human being, can protect against cells carrying said carbohydrate structure. In the case of tumor-associated carbohydrate antigens such as TFα, vaccination against and treatment of cancer comprising cells carrying said carbohydrate antigen is possible with these glycosylated glycophorin peptides. Therefore, based on this experiment, GPA-derived TF-α glycosylated peptides are proposed for the use of treating or preventing cancer in human.

### Example 9

### Presentation of GPA26-43 is Dependent on the Localization of the Attached TFα-Structures

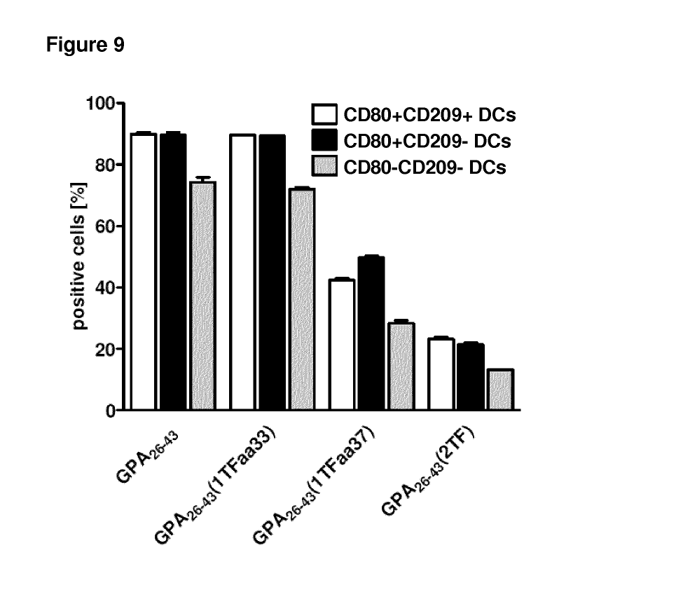

The peptide GPA26-43(2TF) was found to be presented on DCs. By using four different glycosylation variants of GPA26-43, it was aimed to investigate the effect of TFα-glycosylation on the presentation in more detail. GPA26-43(1TFaa33) and the unglycosylated GPA26-43 were presented at a similar percentage, whereas GPA26-43(1TFaa37) was less effectively presented than GPA26-43(1TFaa33) and GPA26-43, but more effectively than GPA26-43(2TF) (FIG. 9). These findings demonstrate that the localization of TFα within the peptide sequence is of relevance for the presentation on DCs. Furthermore, they show that YAATPRAHE or AATPRAHEV is a binding epitope, because a glycan moiety within the MHC binding motif would be more likely to have an effect on binding and presentation than a glycan structure outside this epitope.

### Example 10

### GPA-Derived TF-α Glycosylated Peptides can Activate TFα-Specific T Cells

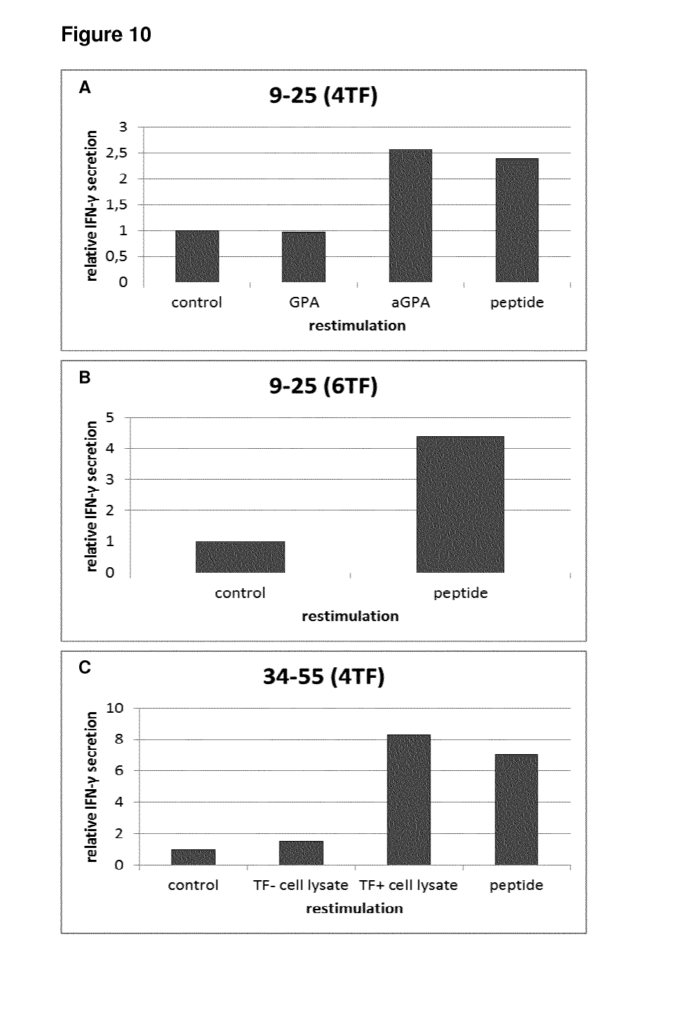

For further demonstrating that DCs loaded with GPA-derived TF-α glycosylated peptides can activate TFα-specific T cells, T cells were primed with DCs loaded with different TF-α glycosylated GPA peptides and restimulated. Priming was performed with GPA9-25(4TF), GPA9-25(6TF) and GPA34-55(4TF) loaded DCs. The primed T cells were then restimulated with DCs loaded with the same peptide, with a lysate of TFα-positive human cells, or with asialoglycophorin A. No restimulation and restimulation with DCs loaded with a cell lysate of TFα-negative human cells or sialylated glycophorin A was used as a control. T cell activation upon restimulation was detected by an IFN-γ ELISPOT assay. Restimulation with TF positively loaded DCs resulted in a strong activation of T cells compared to control (FIG. 10). Therefore, priming with DCs loaded with GPA-derived TF-α glycosylated peptides effectively activated T cells against TF-α. The results further support the finding that glycosylated glycophorin peptides are capable of inducing a cellular immune response against their carbohydrate structure.

1. A glycosylated glycophorin peptide comprising
(i) at least one amino acid independently selected from serine and threonine which carries a carbohydrate structure; and
(ii) at least 5 consecutive amino acids which are identical to or have at least 75% homology to an amino acid segment of the same length within the amino acid sequence of SEQ ID NO: 1.

2. The glycosylated glycophorin peptide according to embodiment 1, having a length of 5 to 50 amino acids.

3. The glycosylated glycophorin peptide according to embodiment 1, having a length of 8 to 30 amino acids.

4. The glycosylated glycophorin peptide according to any one of embodiments 1 to 3, comprising at least 8 consecutive amino acids which are identical to or have at least 75% homology to an amino acid segment of the same length within the amino acid sequence of SEQ ID NO: 1.

5. The glycosylated glycophorin peptide according to any one of embodiments 1 to 4, wherein said consecutive amino acids are identical to or have at least 75% homology to an amino acid segment of the same length within positions 1 to 67 of the amino acid sequence of SEQ ID NO: 1, in particular within positions 21 to 45 of the amino acid sequence of SEQ ID NO: 1.

6. The glycosylated glycophorin peptide according to any one of embodiments 1 to 5, wherein the entire amino acid sequence of the glycophorin peptide is identical to or has at least 75% homology to an amino acid segment of the same length within the amino acid sequence of SEQ ID NO: 1

7. The glycosylated glycophorin peptide according to any one of embodiments 1 to 6, wherein the entire amino acid sequence of the glycophorin peptide is identical to or has at least 75% homology to an amino acid segment of the same length within positions 1 to 67 of the amino acid sequence of SEQ ID NO: 1, in particular within positions 21 to 45 of the amino acid sequence of SEQ ID NO: 1.

8. The glycosylated glycophorin peptide according to any one of embodiments 1 to 7, comprising the amino acid sequence of position 34 to 42 of SEQ ID NO: 1 or an amino acid sequence which has at least 85% homology thereto.

9. The glycosylated glycophorin peptide according to any one of embodiments 1 to 7, comprising the amino acid sequence of position 48 to 55 of SEQ ID NO: 1 or an amino acid sequence which has at least 75% homology thereto.

10. The glycosylated glycophorin peptide according to any one of embodiments 1 to 7, comprising or consisting of an amino acid sequence selected from the group consisting of
(i) the amino acid sequence of position 1 to 9 of SEQ ID NO: 1, in particular SEQ ID NO: 2;
(ii) the amino acid sequence of position 9 to 25 of SEQ ID NO: 1, in particular SEQ ID NO: 2;
(iii) the amino acid sequence of position 26 to 43 of SEQ ID NO: 1, in particular SEQ ID NO: 2;
(iv) the amino acid sequence of position 34 to 55 of SEQ ID NO: 1, in particular SEQ ID NO: 2;
(v) the amino acid sequence of position 41 to 55 of SEQ ID NO: 1, in particular SEQ ID NO: 2;
(vi) the amino acid sequence of position 45 to 55 of SEQ ID NO: 1, in particular SEQ ID NO: 2;
(vii) the amino acid sequence of position 48 to 55 of SEQ ID NO: 1, in particular SEQ ID NO: 2;
(viii) the amino acid sequence of position 48 to 63 of SEQ ID NO: 1, in particular SEQ ID NO: 2;
(ix) the amino acid sequence of position 21 to 40 of SEQ ID NO: 1, in particular SEQ ID NO: 2; and
(x) the amino acid sequence of position 21 to 45 of SEQ ID NO: 1, in particular SEQ ID NO: 2; and
(xi) an amino acid sequence which has at least 75% homology to one of the sequences according to (i) to (x).

11. The glycosylated glycophorin peptide according to any one of embodiments 1 to 8, comprising or consisting of the amino acid sequence of position 26 to 43 of SEQ ID NO: 1, wherein the threonine corresponding to position 33 of SEQ ID NO: 1 and/or the threonine corresponding to position 37 of SEQ ID NO: 1 carries a carbohydrate structure.

12. The glycosylated glycophorin peptide according to embodiment 11, comprising exactly one carbohydrate structure, which is attached to the threonine corresponding to position 33 of SEQ ID NO: 1.

13. The glycosylated glycophorin peptide according to embodiment 11, comprising exactly one carbohydrate structure, which is attached to the threonine corresponding to position 37 of SEQ ID NO: 1.

14. The glycosylated glycophorin peptide according to any one of embodiments 1 to 7 and 10, comprising or consisting of the amino acid sequence of position 48 to 63 of SEQ ID NO: 1, wherein the threonine corresponding to position 50 of SEQ ID NO: 1 carries a carbohydrate structure.

15. The glycosylated glycophorin peptide according to any one of embodiments 1 to 7, comprising or consisting of the amino acid sequence of position 1 to 9 of SEQ ID NO: 1, wherein the serine corresponding to position 2 of SEQ ID NO: 1 and/or the threonine corresponding to position 3 of SEQ ID NO: 1 and/or the threonine corresponding to position 4 of SEQ ID NO: 1 carries a carbohydrate structure.

16. The glycosylated glycophorin peptide according to any one of embodiments 1 to 15, wherein the glycosylated glycophorin peptide or a part thereof is capable of binding to MHC class II proteins.

17. The glycosylated glycophorin peptide according to embodiment 16, wherein the glycosylated glycophorin peptide has a length of 15 to 24 amino acids.

18. The glycosylated glycophorin peptide according to embodiment 16 or 17, wherein at least one of the amino acids carrying a carbohydrate structure is part of the MHC class II binding motif.

19. The glycosylated glycophorin peptide according to any one of embodiments 1 to 15, wherein the glycosylated glycophorin peptide or a part thereof is capable of binding to MHC class I proteins and has a length of 5 to 14 amino acids, preferably 8 or 9 amino acids.

20. The glycosylated glycophorin peptide according to any one of embodiments 1 to 19, wherein the carbohydrate structure is a carbohydrate tumor epitope.

21. The glycosylated glycophorin peptide according to any one of embodiments 1 to 20, wherein the carbohydrate structure has a formula selected from the group consisting of
(i) Galβ1,3-GalNAcα1-;
(ii) GalNAcα1-;
(iii) Siaα2,3-Galβ1,3-GalNAcα1-;
(iv) Siaα2,6-GalNAcα1-;
(v) Galβ1,3-(Fucα1,4)-GlcNAcβ1-;
(vi) Siaα2,3-Galβ1,3-(Fucα1,4)-GlcNAcβ1-;
(vii) Galβ1,4-(Fucα1,3)-GlcNAcβ1-;
(viii) Siaα2,3-Galβ1,4-(Fucα1,3)-GlcNAcβ1-;
(ix) Fucα1,2-Galβ1,4-(Fucα1,3)-GlcNAcβ1-;
(x) Fucα1,2-Galβ1,3-(Fucα1,4)-GlcNAcβ1-; and
(xi) Fucα1,2-Galβ1,3-GalNAcβ1,3-Galα1,4-Galβ1,4-Glcβ1-.

22. The glycosylated glycophorin peptide according to any one of embodiments 1 to 21, wherein the carbohydrate structure has the formula Galβ1,3-GalNAcα1-.

23. The glycosylated glycophorin peptide according to any one of embodiments 1 to 21, wherein the carbohydrate structure has the formula GalNAcα1-.

24. A conjugate comprising the glycosylated glycophorin peptide according to any one of embodiments 1 to 23 covalently coupled to another agent.

25. A method for producing antigen presenting cells which present an epitope comprising or consisting of a carbohydrate structure of interest, comprising the step of contacting antigen presenting cells with the glycosylated glycophorin peptide according to any one of embodiments 1 to 23 or the conjugate according to embodiment 24, wherein the glycosylated glycophorin peptide carries the carbohydrate structure of interest.

26. The method according to embodiment 25, wherein the antigen presenting cells are dendritic cells.

27. An antigen presenting cell obtainable by the method according to embodiment 26 or 26.

28. A method for producing activated T cells against a carbohydrate structure of interest, comprising the step of contacting T cells thereof with antigen presenting cells according to embodiment 27.

29. The method according to embodiment 28, wherein the antigen presenting cells according to embodiment 27 are used for priming the T cells or for restimulating the T cells after priming with antigen presenting cells loaded with a different agent carrying the carbohydrate structure of interest.

30. An activated T cell obtainable by the method according to embodiment 28 or 29.

31. The glycosylated glycophorin peptide according to any one of embodiments 1 to 23, the conjugate according to embodiment 24, the antigen presenting cell according to embodiment 27 or the activated T cell according to embodiment 30 for use in medicine.

32. The glycosylated glycophorin peptide according to any one of embodiments 1 to 23, the conjugate according to embodiment 24, the antigen presenting cell according to embodiment 27 or the activated T cell according to embodiment 30 for treating or preventing cancer which is positive for the carbohydrate structure of the glycosylated glycophorin peptide.

33. The glycosylated glycophorin peptide according to any one of embodiments 1 to 23, the conjugate according to embodiment 24, the antigen presenting cell according to embodiment 27 or the activated T cell according to embodiment 30 for vaccination against cancer which is positive for the carbohydrate structure of the glycosylated glycophorin peptide.

34. A glycosylated glycophorin peptide having a length of 8 to 100 amino acids, comprising
(i) at least one amino acid independently selected from serine and threonine which carries a carbohydrate structure; and
(ii) at least 8 consecutive amino acids which are identical to or have at least 75% homology to an amino acid segment of the same length within the amino acid sequence of SEQ ID NO: 1.

35. The glycosylated glycophorin peptide according to claim 34, having a length of 8 to 50, preferably 8 to 25 amino acids.

36. The glycosylated glycophorin peptide according to claim 34 or 35, wherein said consecutive amino acids are identical to or have at least 75% identity to an amino acid segment of the same length within positions 1 to 67 of the amino acid sequence of SEQ ID NO: 1.

37. The glycosylated glycophorin peptide according to claim 36, wherein the entire amino acid sequence of the glycophorin peptide is identical to or has at least 75% identity to an amino acid segment of the same length within positions 1 to 67 of the amino acid sequence of SEQ ID NO: 1.

38. The glycosylated glycophorin peptide according to any one of claims 34 to 37, comprising
(i) the amino acid sequence of position 34 to 42 of SEQ ID NO: 1 or an amino acid sequence which has at least 80% homology thereto; or
(ii) the amino acid sequence of position 48 to 55 of SEQ ID NO: 1 or an amino acid sequence which has at least 80% homology thereto.

39. The glycosylated glycophorin peptide according to any one of claims 34 to 37, comprising or consisting of an amino acid sequence selected from the group consisting of
(i) the amino acid sequence of position 1 to 9 of SEQ ID NO: 1, in particular SEQ ID NO: 2 or SEQ ID NO: 3;
(ii) the amino acid sequence of position 9 to 25 of SEQ ID NO: 1, in particular SEQ ID NO: 2;
(iii) the amino acid sequence of position 26 to 43 of SEQ ID NO: 1, in particular SEQ ID NO: 2;
(iv) the amino acid sequence of position 34 to 55 of SEQ ID NO: 1, in particular SEQ ID NO: 2;
(v) the amino acid sequence of position 41 to 55 of SEQ ID NO: 1, in particular SEQ ID NO: 2;
(vi) the amino acid sequence of position 45 to 55 of SEQ ID NO: 1, in particular SEQ ID NO: 2;
(vii) the amino acid sequence of position 48 to 55 of SEQ ID NO: 1, in particular SEQ ID NO: 2;
(viii) the amino acid sequence of position 48 to 63 of SEQ ID NO: 1, in particular SEQ ID NO: 2;
(ix) the amino acid sequence of position 21 to 40 of SEQ ID NO: 1, in particular SEQ ID NO: 2; and
(x) the amino acid sequence of position 21 to 45 of SEQ ID NO: 1, in particular SEQ ID NO: 2; and
(xi) an amino acid sequence which has at least 75% identity to one of the sequences according to (i) to (x).

40. The glycosylated glycophorin peptide according to claim 35, comprising or consisting of the amino acid sequence of position 26 to 43 of SEQ ID NO: 2, wherein the threonine corresponding to position 33 of SEQ ID NO: 2 and/or the threonine corresponding to position 37 of SEQ ID NO: 2 carries a carbohydrate structure.

41. The glycosylated glycophorin peptide according to claim 35, comprising or consisting of the amino acid sequence of position 48 to 63 of SEQ ID NO: 2, wherein the threonine corresponding to position 50 of SEQ ID NO: 2 carries a carbohydrate structure.

42. The glycosylated glycophorin peptide according to claim 35, comprising or consisting of the amino acid sequence of position 1 to 9 of SEQ ID NO: 2 or 3, wherein the serine corresponding to position 2 of SEQ ID NO: 2 or 3 and/or the threonine corresponding to position 3 of SEQ ID NO: 2 or 3 and/or the threonine corresponding to position 4 of SEQ ID NO: 2 or 3 carries a carbohydrate structure.

43. The glycosylated glycophorin peptide according to any one of claims 34 to 42, wherein the glycosylated glycophorin peptide or a part thereof is capable of binding to MHC class II proteins.

44. The glycosylated glycophorin peptide according to claim 43, wherein at least one of the amino acids carrying a carbohydrate structure is part of the MHC class II binding motif.

45. The glycosylated glycophorin peptide according to any one of claims 34 to 44, wherein the carbohydrate structure has the formula Galβ1,3-GalNAcα1- or the formula GalNAcα1-.

46. A conjugate comprising the glycosylated glycophorin peptide according to any one of claims 34 to 45 covalently coupled to another agent.

47. The glycosylated glycophorin peptide according to any one of claims 34 to 45 or the conjugate according to claim 46 for use in medicine.

48. The glycosylated glycophorin peptide according to any one of claims 34 to 45 or the conjugate according to claim 46 for treating, preventing or vaccinating against cancer which is positive for the carbohydrate structure of the glycosylated glycophorin peptide.


SEQUENCE LISTING (3 sequences)

SEQ ID NO: 1 - protein, 131 aa, Homo sapiens
  SOURCE 1..131: /mol_type=“protein” /organism=“Homo sapiens”
SSTTGVAMHTSTSSSVTKSYISSQTNDTHKRDTYAATPRAHEVSEISVRTVYPPEEETGERVQLAHHFSEPEITLIIFGVMAGVIGTILLISTGIRRLIKKSPSDVKPLPSPDTDVPLSSVEIENPETSDQ

SEQ ID NO: 2 - protein, 131 aa, Homo sapiens; identical to SEQ ID NO: 1
  SOURCE 1..131: /mol_type=“protein” /organism=“Homo sapiens”

SEQ ID NO: 3 - protein, 131 aa, Homo sapiens
  SOURCE 1..131: /mol_type=“protein” /organism=“Homo sapiens”
LSTTEVAMHTSTSSSVTKSYISSQTNDTHKRDTYAATPRAHEVSEISVRTVYPPEEETGERVQLAHHFSEPEITLIIFGVMAGVIGTILLISTGIRRLIKKSPSDVKPLPSPDTDVPLSSVEIENPETSDQ